(19) United States
(12) Patent Application Publication
(10) Pub. No.: US 2006/0171996 A1
(43) Pub. Date: Aug. 3, 2006

(54) Antibacterial pectocellulose

(76) Inventor: Takuo Sakai, Osaka-fu (JP)

Correspondence Address: THE WEBB LAW FIRM, P.C., 700 KOPPERS BUILDING, 436 SEVENTH AVENUE, PITTSBURGH, PA, 15219, US

(21) Appl. No.: 10/549,461

(22) PCT Filed: Mar. 5, 2004
(86) PCT No.: PCT/JP04/02789

§ 371 Date: Sep. 12, 2005

(30) Foreign Application Priority Data
Mar. 13, 2003 (JP) 2003-068837

(51) Int. Cl. A61K 31/717; A61K 9/70; A61K 33/34; A61K 33/38

(52) U.S. Cl. 424/443; 514/57; 424/618; 424/638

(57) ABSTRACT

Antibacterial pectocellulosic fibers or antibacterial pectocellulosic fiber fabrics obtained by treating pectocellulosic fibers or pectocellulosic fabrics with at least one chemical substance selected from the group consisting of acids, bases, salts thereof, chelating agents, and a pectin-degrading enzyme to lower the pectin content in pectocellulosic fibers to 1 to 80% by mass with respect to the pectin content before the treatment and then supporting antibacterial agents comprising ionic inorganic compounds or antibacterial agents comprising ionic organic compounds on the treated pectocellulosic fibers or pectocellulosic fiber fabrics.

The description of this application claims benefit of priority based on Japan Patent Application No.2003-068837, the entire same contents of which are incorporated by reference herein.

### BACKGROUND OF THE INVENTION

1. Field of the Invention

The present invention relates to antibacterial cellulose in which antibacterial agents bond with pectocellulose, particularly in which antibacterial agents chemically bond with pectocellulose, and further particularly, the present invention relates to antibacterial pectocellulosic fibers, pectocellulosic fiber fabrics, and production method thereof in which pectocellulosic fibers or pectocellulosic fiber fabrics support ionic antibacterial agents.

2. Prior Art

Cellulosic fibers including cotton fibers representing natural fibers are mass produced all over the globe and they are precious fibers in that they have good recycling property which is now one of hot issues. Also, cotton fibers in themselves are comfortable fiber materials originally provided with appropriate hygroscopic property, softness and the like without any special improvements. However, recently, incidents caused by various kinds of bacteria frequently occur, including food-poisoning cases of O-157 bacteria, Legionella problems in 24 hour baths, and the like. Further, increase in damp in super-insulated houses, generation of bacteria, molds, ticks, and the like caused by air ventilation shortage are also reported. Under such circumstances, interest for bacteria among consumers has been intensified remarkably. In response to such tendencies, many kinds of antibacterial products are appearing in the market and to be specific, diversified products such as kitchen utensils, goods used for baths and toilets, home electric appliances, household equipments, and the like are targeted for antibacterial processes.

Regarding antibacterial processes for cellulosic fiber products, for example, cotton products, methods of using antibacterial metals such as silver, copper, and the like as antibacterial agents are known. In antibacterial fiber products with silver ion, in many cases, elution-type agents are used which show antibacterial property by elution of silver ion and as carriers of these elution-type agents, zeolite, clay minerals, glasses, and the like are known. In addition, methods of imparting antibacterial property by impregnating mixture liquid of these antibacterial agents and urethane resins with cellulosic fiber products, followed by drying are known. Further, methods of misting mixture liquid including antibacterial agents to fibers using sprays and the like can be exemplified. However, such cellulosic fiber products under conventional antibacterial processes as above have problems that antibacterial agents separate from fibers by relatively few washing frequencies and consequently, antibacterial effects weaken in relatively short time. Further, although methods of imparting antibacterial property to cellulosic fibers by impregnating spinning oleum including methylol resins and cross-linkage catalyzers with cellulosic fibers (Patent document 1/Japan Unexamined patent publication No.2000-355880), methods of imparting antibacterial property bonding metal ions such as silver ions, copper ions, and the like to cotton fibers with polyphenol as spacer(Patent document 2/Japan Unexamined patent publication No.2000-204182),and the like have been proposed, none of them are satisfactory enough in sustaining antibacterial property.

Therefore, methods capable of easily and stably bonding functional materials such as antibacterial agents and the like with fibers in a sustainable manner have been requested. Natural fibers, for example, cotton fibers are polysaccharides whose main constituent is cellulose and have anionic charge derived from a hydroxyl group of glucose sugar, which is one of the components. However, since the charge is very weak, it is impossible to directly bond other functional materials with cotton fibers regardless of whether they are organic or inorganic, and therefore, researches for developing methods of directly bonding functional materials with cotton fibers and the like have been attempted strengthening this negative charge by chemical treatment. Other methods have also been developed in which ceramics in microscopic powdery forms adsorb functional materials such as antibacterial agents, and the like, thereby introducing this into cotton fibers. However, both methods usually require pretreatment for extreme chemical reaction and this pretreatment damages original property of cotton fibers as well as increasing costs, which become important impediment against imparting functionality to cotton fibers. Therefore, methods capable of easily and stably bonding functional materials such as antibacterial agents and the like with cotton fibers in a sustainable manner have been requested.

The object of the present invention is to provide cellulose in which antibacterial agents bond with cellulose derived from natural cellulosic fibers, and the object of the present invention is to provide antibacterial pectocellulosic fibers or pectocellulosic fiber fabrics in which antibacterial agents are supported on pectocellulosic fibers and antibacterial agents stably bond with fibers in a sustainable manner, and antibacterial agents are not easily separated by washing and the like.

### SUMMARY OF THE INVENTION

As a result of intensive studies on the above problems, the present inventor has found that when the methods of bonding ionic pectocellulosic fibers or antibacterial pectocellulosic fabrics with ionic inorganic compound antibacterial agents or organic compound antibacterial agents obtained by treating pectopellulose fibers or pectocellulosic fabrics with at least one chemical substance selected from the group composed of acids, bases, salts thereof, chelating agents, and pectin-degrading enzyme to leave the pectin content in pectocellulosic fibers to 1 to 80% by mass with respect to the pectin content before the treatment and then supporting antibacterial agents comprising ionic inorganic compounds or antibacterial agents comprising ionic organic compounds on the treated pectocellulosics or pectocellulosic fabrics are employed, antibacterial pectocellulose including pectocellulose in which inorganic compound antibacterial agents or organic compound antibacterial agents bond with pectin included in pectocellulose can be provided.

The present invention relates to antibacterial pectocellulose including pectocellulose in which inorganic compound antibacterial agents or organic compound antibacterial agents bond with pectin included in pectocellulose.

As an embodiment of the above bonds, chemical bond is included and ionic inorganic compound antibacterial agents or organic compound antibacterial agents are ionically bound with an active group having ionic bond capacity in pectin included in said pectocellulose. Although said pectocellulose is not specifically limited, pectocellulose derived from materials selected from Japanese paper including kouzo and mitsumata, cotton, hemp, rayon, deccan hemp, and the groups of each of these materials can be used.

In addition, preferable embodiments of the present invention relates to

(1) antibacterial pectocellulosic fibers or antibacterial pectocellulosic fiber fabrics obtained by treating pectopellulosic fibers or pectocellulosic fiber fabrics with at least one chemical substance selected from the group consisting of acids, bases, salts thereof, chelating agents, and a pectin-degrading enzyme to leave the pectin content in pectocellulosics to 1 to 80% by mass with respect to the pectin content before the treatment and then supporting antibacterial agents comprising ionic inorganic compounds or antibacterial agents comprising ionic organic compounds on the treated pectocellulosics or pectocellulosic fabrics,

(2) pectocellulosic fibers or pectocellulosic fiber fabrics as set forth in (1), wherein pectocellulosic fibers or antibacterial pectocellulosic fiber fabrics are composed of cotton or linen,

(3) pectocellulosic fibers or pectocellulosic fiber fabrics as set forth in (1) or (2), wherein acids are inorganic acids such as phosphoric acid, sulfuric acid, and the like or organic acids such as acetic acid and the like, bases are alkalis such as sodium hydrate, potassium hydrate, calcium hydrate, and the like, salts are those composed of these acids and base, chelating agents are ethylenediaminetetraacetic acid, nitrotriacetic acid, and the like,

(4) pectocellulosic fibers or pectocellulosic fiber fabrics as set forth in any one of claims (1) to (3), wherein inorganic antibacterial agents are any of silver, copper, titanium, or compounds thereof, and organic antibacterial agents are quaternary ammonium, chitin, chitosan, and the like,

(5) method of producing antibacterial pectocellulosic fibers or antibacterial pectocellulosic fiber fabrics obtained by treating pectopellulosic fibers or pectocellulosic fiber fabrics with at least one chemical substance selected from the group consisting of acids, bases, salts thereof, chelating agents, and a pectin-degrading enzyme to leave the pectin content of pectocellulosic fibers to about 1 to 80% by mass with respect to the pectin content before the treatment and then supporting antibacterial agents comprising ionic inorganic compounds or antibacterial agents comprising ionic organic compounds on the treated pectocellulosic fibers or pectocellulosic fabrics, and

(6) fiber products composed of pectocellulosic fibers set forth in any of (1) to (4).

### PREFERRED EMBODIMENT IN CARRYING OUT THE INVENTION

Hereinafter, most preferred embodiment for carrying out the present invention is explained.

The present invention relates to antibacterial pectocellulose which includes pectocellulose in which inorganic compound antibacterial agents or organic compound antibacterial agents are bound with pectin which is included in pectocellulose. As said inorganic antibacterial agents, silver, copper, or titanium or metallic compounds including them are preferable and said organic antibacterial agents are preferably quaternary ammonium, chitin, or chitosan.

As embodiments of the present invention, antibacterial pectocellulosic fibers composed of said antibacterial pectocellulose can be provided, however, regardless of the forms of “fibers”, they can be provided as antibacterial composite cellulose.

Therefore, as embodiments of the present invention, antibacterial pectocellulose itself or each kind of fiber products including such antibacterial cellulosic fibers can be provided and further, fiber products in which said antibacterial pectocellulosic fibers are included alone or in combinations or complexed with other fibers can be provided.

Specific embodiments of the present invention relate to antibacterial pectocellulosic fibers or pectocellulosic fiber fabrics obtained by treating pectocellulosic fibers or pectocellulosic fiber fabrics with at least one chemical substance selected from the group consisting of acids, bases, salts thereof, chelating agents, and a pectin-degrading enzyme to leave the pectin content of pectocellulosic s to about 1 to 80% by mass with respect to the pectin content before the treatment and then supporting antibacterial agents comprising ionic inorganic compounds or antibacterial agents comprising ionic organic compounds on the treated pectocellulosic fibers or pectocellulosic fiber fabrics.

Methods of quantifying pectin in the present invention are conducted by the following. Pectocellulosic fibers or pectocellulosic fiber fabrics are heated for 60 minutes at a temperature of 90° C. in 0.1 M sodium hydrate solution and the content of galacturonic acid in said solution is measured by carbazole-sulfuric acid method, thereby defining this as the content of pectin. To be more specific, 0.125 ml of subjected liquid treated with sodium hydrate as above and 0.125 ml of 0.2% by mass of carbazole solution (ethanol solution) is mixed, to which 1.5 ml of 31.5 N sulfuric solution is ice-cooled, added, and is thoroughly mixed. Next, this mixture solution is heated for 20 minutes at a temperature of 75° C., followed by being left to cool until it gets to the room temperature, and absorbance at the wavelength of 570 nm is measured with spectrophotometer, and from this absorbance, the content of galacturonic acid in subjected liquid is scanned from the standard curve prepared by measuring the known content of galacturonic acid separately, and from the scanned numerical values, the content of pectin in pectocellulosic fibers is calculated.

Pectocellulosic fibers in the present invention mean natural fibers which include pectin and pectocellulose means cellulose which includes pectin. In short, any kinds can be used as long as they are fibers or cellulose which include pectin. As pectocellulosic fibers, for example, cotton, linen, cellulosic fibers such as rayon and the like can be exemplified and among them, cotton is preferable. In addition, as mentioned above, Japanese paper including kouzo and mitsumata, or deccan hemp can be used. Or each embodiment of materials thereof can be used, too. Or mixture of at least two of these can be used, too. Although pectocellulosic fibers obtained from natural ingredients vary depending on kinds and production areas, usually, cotton includes about 7 to 8% by mass of pectin and linen includes about 10 to 11% by mass of pectin.

Acids to be used in the present invention may either be inorganic acids or organic acids. As inorganic acids, they are not specifically limited and examples include phosphoric acid, sulfuric acid, nitric acid, sulfonic acid, hydrochloric acid, boric acid, and the like and among them, phosphoric acid and sulfuric acid are preferable. As organic acids, they are not specifically limited and examples include acetic acid, butyric acid, carboxylic acid, lactic acid, formic acid, oxalic acid, tartaric acid, citric acid, malic acid, sulfamic acid, pyruvic acid, and the like and among them, acetic acid and butyric acid are preferable.

As bases, they are not specifically limited and examples include sodium hydrate, potassium hydrate, calcium hydrate, potassium carbonate, adenine and the like and among them, sodium hydrate, potassium hydrate, and calcium hydrate are preferable.

As salts, any kind thereof can be used as long as they are formed by above mentioned acids and bases, and sodium sulfate, potassium sulfate, and potassium secondary phosphate are preferable.

As chelating agents, they are not specifically limited and specific examples include ethylenediaminetetraacetic acid or salts thereof, nitrotriacetic acid or salts thereof, citric acid or salts thereof, etidronic acid, L-asparatic acid diacetate, L-glutamic acid diacetate, sodium tripolyphosphate, sodium pyrophosphate, sodium hexametaphosphate, and the like and among them, ethylenediaminetetraacetic acid or salts thereof, nitrotriacetic acid or salts thereof, and sodium hexametaphosphate are preferable.

As pectin-degrading enzymes in the present invention, preferably, protopectinase can be used. Protopectinase is a generic term of enzymes having activity of liberating water-soluble pectin from water-insoluble protopectin present in plant tissue. In the present invention, as pectin-degrading enzymes, microbes producing or including this enzyme or treatment products thereof may be used. Or, as pectin-degrading enzymes, commercially available products may be used.

Specific examples of microbes producing pectin-degrading enzymes used in the present invention are as follows.

1. As microbes which are yeasts, the following are exemplified. As a microbe belonging to Tricosporon, Tricosporon penicillatum; as microbes belonging to Endomyces, Endomyces geotrichum and Endomyces lindneri; as microbes belonging to Endomycopsis, Endomycopsis capsularis, Endomycopsis vernalis; as microbes belonging to Saccharomyces, Saccharomyces uvarum, Saccharomyces bailii, Saccharomyces delbrueckii, Saccharomyces fermentati; as a microbe belonging to Schizosaccharomyces, Schizosaccharomyces octosporus; as microbes belonging to Pichia, Pichia orientalis, Pichia polymorpha, Pichia farinose; as microbes belonging to Hansenula, Hansenula saturnus, Hansenula minuta; as microbes belonging to Debaryomyces, Debaryomyces hansenii, Debaryomyces castellii; as microbes belonging to Hanseniaspora, Hanseniaspora valbyensis, Hanseniaspora uvarum; as microbes belonging to Torulopsis, Torulopsis sphaerica, Torulopsis pinus; as microbes belonging to Candida, Candida krusei, Candida glaebosa, Candida macedoniensis; and as microbes belonging to Kluyveromyces, Kluyveromyces fragilis, Kluyveromyces lactis, Kluyveromyces marxianus, and Kluveromyces drosophilarum, and as microbes similar to these microbes and variants thereof, for example, following strains are exemplified such as Tricosporon penicillatum SNO-3 ATCC 42397, Candida krusei IFO 0013, Candida glaebosa IFO 1353, Candida macedoniensis AKU 4587, Debaryomyces hansenii IFO 0794, Debaryomyces castellii IFO 1359, Endomyces geotrichum IFO 9541, Endomyces lindneri AKU 4206, Hanseniaspara Valbyensis IFO 115, Hanseniaspora uvarum IFO 1413, Hansenula saturnus IFO 0117, Hansenula minuta IFO 0975, Kluyveromyces fragilis IFO 0288, Kluyveromyces lactis IFO 1090, Kluyveromyces marxianus IFO 0277, Kluyveromyces drosophilarum IFO 1012, Pichia orientalis IFO 1279, Pichia polymorpha AKU 4250, Pichia farinosa AKU 4251, Saccharomyces uvarum IFO 0565, Saccharomyces bailii IFO 1047, Saccharomyces delbrueckii IFO 0285, Saccharomyces fermentati IFO 0422, Schizosaccharomyces octosporus IFO 0353, Torulopsis sphaerica IFO 0648, Torulopsis pinus IFO 0741, Endomycopsis capsularis IFO 0672, and Endomycopsis vernalis AKU 4210;

2. As microbes belonging to Bacillus, the following are exemplified. Bacillus subtilis, Bacillus amyloliquefaciens, Bacillus cereus, Bacillus circulans, Bacillus coagulans, Bacillus firmus, Bacillus licheniformis, Bacillus pumilus, Bacillus macerans, and as fungus similar to these strains and variants thereof, for example, following strains are exemplified such as Bacillus subtilis IFO 3108, 3134, 3336, 3513, 12112, 12113, 12210, 13719, 13721, 14117, and 14140; Bacillus amyloliquefaciens IFO 14141, Bacillus cereus IFO 3002 and 3132, Bacillus circulans IFO 13632, Bacillus coagulans IFO 12583, Bacillus firmus IFO 3330, Bacillus licheniformis 14206, Bacillus pumilus IFO 12087, and Bacillus macerans IFO 3490.

3. As microbes of filamentous fungus, the following are exemplified. Galactoomyces reessii L, Aspergillus oryzae, Aspergillus sojae, Rhizopus oryzae, Trametes sanguinea, Trametes orientalis, Trametes albida, Trametes cubensis, Trametes cinnabarina, Trametes gibbosa, Trametes kusanoana, Trametes serialis, and as fungus similar to these strains and variants thereof, for example, following strains are exemplified such as Galactoomyces reessii L. IAM 129, Trametes sanguinea IFO 6490, 6491, Trametes orientalis IFO 6483, 6484, Trametes albida IFO 6434, 6510, Trametes cubensis IFO 9285, Trametes gibbosa IFO 4946, Trametes Kusanoana IFO 6264, Trametes serialis IFO 9286, Aspergillus oryzae IFO 4277, Aspergillus sojae IFO 4200, and Rhizopus oryzae IFO 4734.

Among the above mentioned pectin-degrading enzyme-producing fungus, Kluyveromyces marxianus (IFO 0277), Kluyveromyces fragilis (IFO 0288), Tricosporon penicillatum SNO-3 (ATCC 42397), Galactoomyces reessii L. (IAM 129), Bacillus subtilis (IFO 12113), Bacillus subtilis (IFO 3134), or Trametes sanguinea (IFO 6490) are preferable.

Enzymes used in the present invention are obtained by culturing the above microbes by common procedure and treating them. Although culturing conditions are not necessarily identical depending on microbes used, the conditions are appropriately determined so that the production amount of enzymes gets maximum. Medium used for culture are not specifically limited and any medium with each kind of nutritional sources added which are commonly used for ordinary culture can be used. As commonly used medium, starch, peptone, casein hydrolysate, yeast extract, glucose sugar, or depending on the case, inorganic salts such as phosphate, magnesium salt, and potassium salt and the like can appropriately be used. In addition, nutritional sources such as wheat bran or soy bean powders may be added.

Although culturing conditions of microbes on such medium are appropriately determined so that the targeted enzyme production gets maximum, usually, microbes are cultured for about 10 to 50 hours at about 20 to 37° C. Culture may be any of shaking culture, static culture, aerated spinner culture or culture.

Although cultured liquid obtained by the above methods can be treated by immersing pectocellulosic fibers or pectocellulosic fiber fabrics, enzyme liquid which excludes all or some solid contents such as fungus bodies and the like from cultured liquid by centrifugation, filtration, dialysis and the like can preferably be used. In addition, enzyme liquid in which enzymes obtained by purifying by such common methods as column chromatography and the like are diluted to appropriate concentration may be used as well. Further, to enzyme liquid, substances promoting pectin degradation action including inorganic salts, surfactants, and the like may be added.

In pectocellulosic fibers, other than cellulose, so-called impurities such as wax, pectin, protein, and the like are included, which prevent pectocellulosic fibers from getting hydrophilic. Therefore, under the name of scouring, for example, such methods as treatment under high temperature (about not less than 90° C.) are employed immersing pectocellulosic fibers or pectocellulosic fiber fabrics in mixture of scouring auxiliary agents whose main ingredient is a surfactant generally together with alkali, by which impurities included in pectocellulosic fibers are completely removed thereby being provided for practical use.

The present invention has characteristics in that it focuses attention on the fact that pectin which possesses most of the impurities contained in pectocellulosic fibers is acidic polysaccharide and has high reactivity, and without completely removing pectin contained in pectocellulosic fibers and without damaging hydrophilic property of pectocellulosic fibers, the present invention treats pectocellulosic fibers or pectocellulosic fiber fabrics with chemical substances selected at least one from the group consisting of bases, salts thereof, chelating agents, and pectin-degrading enzymes so that pectin content is about 1 to 80% by mass with respect to the pectin content before treatment and active groups with ionic bond capacity is generated in pectin.

As required, pectocellulosic fibers or pectocellulosic fiber fabrics may be washed by distilled water or acid after treating them with at least one chemical substance selected from the group consisting of acids, bases, salts thereof, chelating agents, and pectin-degrading enzymes. Next, said pectocellulosic fibers or pectocellulosic fiber fabrics are dried thereby obtaining treated pectocellulosic fibers or pectocellulosic fiber fabrics (pectocellulosic fibers or pectocellulosic fiber fabrics having active groups with ionic bond capacity).

Regarding treatment condition by any of acid, base, salts thereof, or chelating agents, concentration of these compounds for treatment is usually about 0.01 to 100 mM, preferably about 0.1 to 50 mM, treatment temperature is usually about 5 to 40° C., preferably about 15 to 25° C., treatment time is usually about 0.1 to 5 hours, preferably about 1 to 2 hours. When pectocellulosic fibers or pectocellulosic fiber fabrics are treated by pectin-degrading enzymes, they are treated by immersing them as they are in cultured liquid of pectin-degrading enzymes obtained by the above methods, however, it is preferable that cultured liquid of pectin-degrading enzymes is treated by using pectin-degrading enzyme liquid in which all or some of solid contents such as fungus bodies and the like are removed by centrifugation, filtration, dialysis, and the like. In addition, pectin-degrading enzyme liquid diluted to appropriate concentration obtained by the common methods such as column chromatography refining methods and the like may be used. Further, to pectin-degrading enzyme liquid, substances which promote pectin degrading effect, the above mentioned salts (preferably inorganic salts) surfactants such as cation surfactants, anionic surfactants, or nonionic surfactants and the like may be added. Concentration of pectin-degrading enzyme added is usually about 1 to 5000 unit/ml (aqueous solution) and preferably, about 1000 to 3000 unit/ml (aqueous solution). Here, 1 unit of pectin-degrading enzyme is defined as enzyme amount which degrades albedo layer of lemon peelings and liberates pectin equivalent to 1 amol of galacturonic acid in 1 hour. Regarding treatment condition of treating pectocellulosic fibers or pectocellulosic fiber fabrics by pectin-degrading enzyme, treatment time is usually about 0.5 to 24 hours, preferably about 2 to 10 hours, pH of pectin-degrading enzyme aqueous solution is usually about 5 to 10, immersing treatment temperature is usually about 30 to 55° C., preferably about 30 to 40° C. For regulating pH, as aqueous solution, buffer solutions such as phosphate buffer solution and the like may be used.

Next, an antibacterial agent is supported on treated pectocellulosic fibers or pectocellulosic fiber fabrics obtained by the above methods (pectocellulosic fibers or pectocellulosic fiber fabrics with active groups with ionic bond capacity). As antibacterial agents, any of inorganic antibacterial agents or organic antibacterial agents maybe used.

As inorganic antibacterial agents, for example, specifically, metal ions such as silver bromide or iodine complex, or silver, copper, zinc, platinum, nickel, cobalt, chrome, titanium and the like, metallic compounds such as oxide of such metals, hydroxide and the like are exemplified. Among them, silver ion as metal ion is preferable. Such inorganic antibacterial agents can be used alone or in combinations of plural of them.

As organic antibacterial agents, for example, specifically, quaternary ammonium, thiapendazole, biazine, polycation such as chitin or chitosan, and the like are exemplified. Among them, quaternary ammonium and chitosan are preferable. These organic antibacterial agents can be used alone or in combinations of plural of them.

When pectocellulosic fibers or pectocellulosic fiber fabrics are treated by antibacterial agents, as treatment condition, concentration of antibacterial agents is usually about 0.1 to 100 mM, preferably, about 1 to 30 mM, treatment temperature is usually about 5 to 40° C., preferably about 15 to 25° C., treatment time is usually about 0.1 to 5 hours, preferably about 1 to 2 hours.

Methods of manufacturing fabrics using antibacterial pectocellulosic fibers with antibacterial agents of the present invention supported are not specifically limited and publicly known methods may be used.

Fabrics can be obtained by for example, turning antibacterial pectocellulosic fibers with antibacterial agents supported to plain weave, twill weave, sateen weave, weft weave, leno weave, oblique weave, and the like. Knitted works can be obtained by for example, turning antibacterial pectocellulosic fibers with antibacterial agents supported to weft stitch such as plain stitch, rubber stitch, purl stitch, and the like, or to warp stitch such as single denbigh stitch and the like, or lace stitch, and the like. Further, by using pectocellulosic fibers with antibacterial agents not supported, fabrics may be manufactured and antibacterial agents may be supported on these fabrics by the present invention. In this case, likewise, methods for manufacturing fabrics are not specifically limited and publicly known methods may be used. As fabrics, the above mentioned materials are exemplified.

Pectocellulosic fibers or pectocellulosic fiber fabrics with antibacterial agents of the present invention supported obtained by the above methods can be used for fiber products for various uses. In addition, pectocellulose of the present invention can be provided as antibacterial cellulose materials.

Examples of such fiber products of the present invention include clothes; household groceries such as handkerchiefs, accessories, ribbons, towels, dish cloths, wiping cloths (shoe rags, floor polishing rags, eye glass cleaning cloths, and the like); bed clothes such as blankets, sheets, bed covers, pillow cases, beddings, cushions, and the like; home furnishing such as carpets, curtains, wall papers, and the like, medical materials such as gauzes, masks, caps, and the like; hobby kits such as handicraft materials and the like. Fiber products of the present invention may be composed of pectocellulosic fiber fabrics alone with the antibacterial agents supported or they may be used for a part of fabric materials. In addition, fiber products of the present invention may be composed of pectocellulosic fibers alone with antibacterial agents of the present invention supported.

### EXAMPLE

Hereinafter, the present invention is explained based on Examples in more specific, however, the present invention is not limited to these.

### Example 1

Non-scoured cotton yukata fabrics (local origin of cotton: Pakistan, cotton thread types used: type 20 (warp and weft), weave types: plain weave, sample sizes: 8 cm in width×8 cm in length, mass: 0.68 g) was treated by pectin-degrading enzyme. (This treatment is called bio-scouring). In other words, said sample was immersed in solution composed of 3000 units/ml of pectin-degrading enzyme (aqueous solution) and 0.1% by mass of a surfactant (UOMIN TE, manufactured by TOKAISEIYU KOGYO K.K) under the conditions of being at a room temperature and with a treatment time of 2 hours (hereinafter, a cloth under such treatment is called a bio-scoured cloth). This bio-scoured cloth was thoroughly water washed and was dried (hereinafter, this cloth is called an ionized cloth). This ionized cloth was put in 50 ml of distilled water and after it was stirred, pH of this distilled water was measured, which was 5.2. An ionized cloth taken out from distilled water was put in a glass container, to which solution of silver nitrite was added to make final concentration 10 mM and reacted at a room temperature for 1 hour. Thus, the ionized cloth with solution of silver nitrate treatment (hereinafter called a silver treatment cloth) was taken out from solution, followed by wringing well to remove solution, further followed by cleansing well with distilled water. As a result of measuring the amount of silver ion bound to a cloth calculated from the formula of pH=−logH+ out of the difference of the pH of cleansing water with this distilled water and the calculated pH (5.2) as above, it was found that about 6 m mol of silver ion was bound to the silver treatment cloth of the present invention. As a result that pectin content in cotton fibers of a bio-scoured cloth was measured based on the above pectin quantitative measuring method, it was found that the pectin content was 3.4% by mass with respect to cotton fibers.

As comparison, separately, a cloth was prepared in which the above non-scoured cotton yukata fabrics (local origin of cotton: Pakistan, cotton thread types used: type 20 (warp and weft), weave types: plain weave, sample sizes: 8 cm in width×8 cm in length, content: 0.68 g) was under heat treatment at a temperature of 90° C. for 1 hour in 0.1 N sodium hydride solution (hereinafter, it is called a chemically scoured cloth). To this chemically scoured cloth, treatment with solution of silver nitrate was conducted as in the case of the bio-scoured cloth (this is called a comparative silver treatment cloth) Like in the case of a silver treatment cloth of the present invention, as a result of calculating the content of silver ion in a comparative silver treatment cloth, bond of silver ion was not recognized in a comparative silver treatment cloth. For information, as a result that pectin content in the cotton fibers of a chemically scoured cloth was measured based on the above pectin quantitative method, pectin content was 0% by mass with respect to cotton fibers.

Further, as a result of measuring bonding amount of silver ion on the silver treatment cloth and the comparative silver treatment cloth of the present invention using an X-ray fluorescence spectrometer (manufactured by Shimadzu Corporation), the amount was not greater than 6.5 m mol and 1 m mol, respectively.

In order to evaluate antibacterial property of a silver treatment cloth and a comparative silver treatment cloth of the present invention, the following tests were conducted. That is, Pseudomonas aeruginosa was cultured by putting a silver treatment cloth sample and a comparative silver treatment cloth sample of the present invention prepared by the above method (sample size: 2.5 cm×2.5 cm each, sample mass: 0.08 g each) to 5 ml of each of two cultured media (containing 2% by mass of glucose, 0.5% by mass of peptone, and 0.5% by mass of yeast extract, hereinafter called GYP cultured media) to which Pseudomonas aeruginosa was added (as culturing condition, at a temperature of 30° C., culturing time: 24 hours). After culture, 1 ml of each culture liquid was taken, followed by diluting to 5 times with distilled water respectively, and each absorbance (at 660 nm) was measured, thereby making numerical values of each absorbance indices for antibacterial property respectively. The results of measured absorbance of a silver treatment cloth and a comparative silver treatment cloth were shown in Table 1. As seen from Table 1, when a silver treatment cloth was added to a culture medium, numerical values of absorbance were small and growth of Pseudomonas aeruginosa was not recognized, showing clearly that the silver treatment cloth of the present invention had antibacterial property. On the other hand, in the case of a comparative silver treatment cloth, numerical values of absorbance were large and proliferation of Pseudomonas aeruginosa was observed, showing that no antibacterial property in a comparative silver treatment cloth and advantage of a silver treatment cloth of the present invention was proved.

Next, in order to evaluate stability of antibacterial property, changes of antibacterial property by repeated water washing of a silver treatment cloth of the present invention were observed. That is, after putting a silver treatment cloth of the present invention in the above Pseudomonas aeruginosa cultured medium as mentioned above to culture it under the above conditions, a silver treatment cloth of the present invention was taken out and was washed for 1 hour in 10 ml of distilled water, and then, this was further put in Pseudomonas aeruginosa cultured medium prepared newly again, followed by culturing under the above condition and absorbance of cultured liquid was measured again as in the above mentioned method.

A series of operation was repeated five times. The results were shown in Table 2. As seen from Table 2, the value of absorbance showed that in a silver treatment cloth of the present invention, antibacterial property was found to be stable without any decrease in antibacterial property even after water washing of five times.

TABLE 1

| | Absorbance at 660 nm *) |
|---|---|
| Silver treatment | 0.069 |
| cloth (present | |
| invention) | |
| Comparative | 2.905 |
| silver | |
| treatment cloth | |

*) The degree of proliferation of Pseudomonas aeruginosa was evaluated by measuring absorbance at 660 nm with a spectrophotometer (Hereinafter, the same).

TABLE 2
Effects on antibacterial property by water washing of a
silver treatment cloth

| Number of | |
|---|---|
| water washing | Absorbance at 660 nm |
| 0 | 0.069 |
| 1 | 0.064 |
| 2 | 0.068 |
| 3 | 0.061 |
| 4 | 0.069 |
| 5 | 0.064 |

### Example 2

Non-scoured cotton yukata fabrics (local origin of cotton: Pakistan, cotton thread types used: type 20 (warp and weft), weave types: plain weave, sample sizes: 8 cm in width×8 cm in length, mass: 0.68 g) was immersed in 0.5 M solution of sodium hexametaphosphoric acid with 0.1% bymass of surfactants (UOMIN TE, manufactured by TOKAI SEIYU KOGYO K.K) added, and was under heat treatment at a temperature of 80° C. for 1 hour, sometimes stirring. After they were thoroughly water washed and dried, as in Example 1, these fabrics were treated with silver nitrate. As in the Example 1, with the method of measuring pH difference, silver ion content in the above sample treated with silver nitrate as above was calculated and the result showed that 7 m mol of silver ion was bound. In addition, as a result that the pectin content in a cotton fiber of above cotton fabric. treated with 0.5 M solution of sodium hexametaphosphoric acid in accordance with the above pectin quantitative measuring method, the pectin content was 6.4% by mass with respect to a cotton fiber.

### Example 3

Non-scoured cotton yukata fabrics (local origin of cotton: Pakistan, cotton thread types used: type 20 (warp and weft), weave types: plain weave, sample sizes: 8 cm in width×8 cm in length, mass: 0.68 g) was immersed in 0.02 M solution of potassium secondary phosphate with 0.1% by mass of surfactants (UOMIN TE, manufactured by TOKAI SEIYU KOGYO K. K) added, and was under heat treatment at a temperature of 80° C. for 1 hour, sometimes stirring. After they were thoroughly water washed and dried, as in Example 1, these fabrics were treated with silver nitrate. As in the Example 1, with the method of measuring pH difference, silver ion content in the above sample treated with silver nitrate as above was calculated and the result showed that 10 m mol of silver ion was bound. In addition, as a result that the pectin content in a cotton fiber of the above cotton fabric treated with 0.02 M solution of potassium secondary phosphate in accordance with the above pectin quantitative measuring method, the pectin content was 5.9% by mass with respect to a cotton fiber.

### Example 4

As in Example 3, 0.6 g of a sample with treatment of silver nitrate was stirred with 100 ml of distilled water and water washing was repeated, and as in Example 1, stability in antibacterial property was evaluated, and as a result, no decrease in antibacterial property by water washing with 5 times was not recognized (Table 3).

TABLE 3
Effects on antibacterial property by water washing

| Number of | |
|---|---|
| water washing | Absorbance at 660 nm |
| 0 | 0.071 |
| 1 | 0.066 |
| 2 | 0.067 |
| 3 | 0.061 |
| 4 | 0.070 |
| 5 | 0.060 |

### Example 5

Stability in antibacterial property was evaluated as in Example 4 with the exception that instead of washing 0.6 g of a sample with treatment of silver nitrate in distilled water for 1 hour as in Example 3, washing was repeated for 1 hour at a temperature of 80° C. in 100 ml of soap liquid with 0.1% by mass of a dishwasher PUREBASE manufactured by TAMANOHADA SOAP CORPORATION (mildlyalkaline, containing 28% of pure soap), and as a result, decrease in antibacterial property by water washing with at least 5 times was not recognized(Table 4).

TABLE 4
Effects on antibacterial property by washing

| Number of | |
|---|---|
| washing | Absorbance at 660 nm |
| Comparison | 2.812 |
| (before silver | |
| treatment) | |
| 0 | 0.108 |
| 1 | 0.134 |
| 2 | 0.122 |
| 3 | 0.110 |
| 4 | 0.133 |
| 5 | 0.125 |

### Example 6

10 m mol of copper ion was observed in cotton knitted or woven fabric when the copper ion content was measured by the same method of pH difference as in Example 1, after treating cotton knitted or woven fabric with copper sulfate by the same treatment method as in Example 1 with the exception that copper sulfate was used instead of silver nitrate and that cotton knitted or woven fabrics (cotton thread types used: type 20, knitting type: plain stitch, sample size: 8 cm in width×8 cm in length) were used instead of unscoured cotton yukata fabrics.

In addition, as a result that the pectin content in a cotton fiber of the above cotton knitted or woven fabric which was bio-scoured as in the Example 1 was measured in accordance with the above pectin quantitative measuring method, the pectin content was 3.1% by mass with respect to a cotton fiber.

### Example 7

The same treatment was conducted as in the Example 6 except that chitosan (chitosan 10 B, manufactured by FUNAKOSHI CO., LTD) was used instead of copper sulfate. Bonding of chitosan equivalent to 5 m mol of glucosamine was observed in a cotton knitted or woven fabric (quantity of chitosan was determined by Elson-Morgan method). In addition, as a result that the pectin content in a cotton fiber of the above cotton knitted or woven fabric which was bio-scoured as in the Example 1 was measured in accordance with the above pectin quantitative measuring method, the pectin content was 3.2% by mass with respect to a cotton fiber.

### Example 8

The same treatment was conducted as in the Example 3 except that 1 g of type 20 cotton thread (Pakistan cotton) was used instead of cotton yukata fabrics. As a result that silver ion content was calculated by the method of measuring pH difference, bonding of 6 m mol of silver ion was observed in a type 20 cotton thread. In addition, as a result that the pectin content in the above cotton thread treated with 0.02 M solution of potassium secondary phosphate was measured in accordance with the above pectin quantitative method, the pectin content was 6.0% by mass with respect to a cotton fiber.

### INDUSTRIAL APPLICABILITY

As mentioned above, antibacterial agents of the present invention are supported firm on pectocellulosic fibers and the antibacterial agents bond with pectocellulosic fibers stably and sustainably, which do not easily separate from pectocellulosics by washing. Therefore, since they can be used for antibacterial pectocellulosic fibers or antibacterial pectocellulosic fiber fabrics they can be used as clothes; household groceries such as handkerchiefs, accessories, ribbons, towels, dish cloths, wiping cloths (shoe rags, floor polishing rags, eye glass cleaning cloths, and the like), short split curtains, and the like; bed clothes such as blankets, sheets, bed covers, pillow cases, beddings, cushions, and the like; home furnishing such as carpets, curtains, wall papers, and the like, medical materials such as gauzes, masks, caps, and the like; hobby kits such as handicraft materials and the like. In addition, they can be used as antibacterial celluloses themselves.

1. Antibacterial pectocellulose comprising pectocellulose in which inorganic compound antibacterial agents or organic compound antibacterial agents are bound with pectin included in the pectocellulose.

2. Antibacterial pectocellulose as set forth in claim 1, wherein ionic inorganic compound antibacterial agents or organic compound antibacterial agents are jonically bound to an active group having an ionic bond capacity in pectin included in said pectocellulose.

3. Antibacterial pectocellulose as set forth in claim 1, wherein said inorganic antibacterial agents are silver, copper, or titanium, or metallic compounds thereof, and said organic antibacterial agents are quaternary ammonium, chitin, or chitosan.

4. Antibacterial pectocellulose as set forth in claim 1, wherein said pectocellulose is derived from materials selected from the group of Japanese paper including kouzo and mitsumata, cotton, linen, rayon, deccan hemp, and each material thereof.

5. Antibacterial pectocellulosic fibers composed of antibacterial pectocellulose set forth in claim 1.

6. Fiber products comprising antibacterial pectocellulosic fibers set forth in claim 5.

7. Fiber products as set forth in claim 6, wherein said antibacterial pectocellulosic fibers are included alone or are mixed or compounded with other fibers.

8. Antibacterial pectocellulosic fibers or pectocellulosic fiber fabrics obtained by treating pectocelluiosic fibers or pectocellulosic fabrics with at least one chemical substance selected from the group consisting of acids, bases, salts thereof, chelating agents, and a pectin-degrading enzyme to lower the pectin content in pectocellulosic fibers to 1 to 80% by mass with respect to the pectin content before the treatment and then supporting antibacterial agents comprising ionic inorganic compounds or antibacterial agents comprising ionic organic compounds on the treated pectocellulosic fibers or pectocellulosic fiber fabrics.

9. Pectocellulosic fibers or pectocellulosic fiber fabrics as set forth in claim 8, wherein said pectocellulosic fibers or said pectocellulosic fiber fabrics are composed of cotton or linen.

10. Pectocellulosic fibers or pectocellulosic fiber fabrics as set forth in claim 8, wherein said acids are phosphoric acid, sulfuric acid or acetic acid, said bases are sodium hydrate, potassium hydrate, or calcium hydrate, said salts are those compose of these acids and bases, and said chelating agents are ethylenediaminetetraacetic acid or nitrotriacetic acid.

11. Pectocellulosic fibers or pectocellulosic fiber fabrics as set forth in claim 8, wherein said inorganic antibacterial agents are silver, copper, or titanium, or metallic compounds thereof, and said organic antibacterial agents are quaternary ammonium, chitin, or chitosan.

12. Method of producing antibacterial pectocellulosic fibers or antibacterial pectocellulosic fiber fabrics obtained by treating pectocellulosic fibers or petcto cellulosic fiber fabrics with at least one chemical substance selected from the group consisting of an acid, a base, salts thereof, a chelating agent, and a pectin-degrading enzyme to lower the pectin content in pectocellulosic fibers to 1 to 80% by mass with respect to the pectin content before the treatment and then supporting antibacterial agents comprising ionic inorganic compounds or antibacterial agents comprising ionic organic compounds on the treated pectocellulosic fibers or pectocellulosic fiber fabrics.

13. Fiber products comprising pectocellulosic fibers set forth in claim 8.
